(19) United States
(12) Patent Application Publication
(10) Pub. No.: US 2005/0215815 A1
(43) Pub. Date: Sep. 29, 2005

(54) Method of producing trimellitic acid

(76) Inventors: Hang-Duk Roh, Gyeonggi (KR); Jong-In Lee, Gyeonggi (KR); Hyun-Sup Shim, Seoul (KR); Byung-Hui Kim, Gyeonggi (KR); Jun-Sang Cho, Gyeonggi (KR); Yoon-Seo Lee, Gyeonggi (KR)

Correspondence Address: Abelman Frayne & Schwab, 150 East 42nd Street, New York, NY, 10017-5612, US

(21) Appl. No.: 10/520,540

(22) PCT Filed: Jan. 7, 2003
(86) PCT No.: PCT/KR03/00023

§ 371 Date: Jan. 4, 2005

(30) Foreign Application Priority Data
Jul. 30, 2002 (KR) 10-2002-0044946

(51) Int. Cl. C07C 51/255

(52) U.S. Cl. 562/412

(57) ABSTRACT

Disclosed is a method of producing trimellitic acid through the liquid-phase oxidation of pseudocumene in acetic acid. The oxidation comprises a) conducting a first oxidation using an initial oxidizing catalytic system at 120-200ᵢÉ for 5-20 min in an oxidizing reactor, said initial oxidizing catalytic system comprising at least three compounds selected from the group consisting of cobalt compound, manganese compound, zirconium compound and bromine compound; b) conducting a second oxidation in situ at 160-220ᵢÉ for 30-60 min under addition of an additional catalytic system, said additional catalytic system comprising at least two compounds selected from the group consisting of cobalt compound, manganese compound, zirconium compound, and bromine compound; and c) completing the oxidation of pseudocumene at a temperature from 180 to 230ᵢÉ for a time from 5 to 20 min without the addition of catalysts into the reactor. The pressure is adjusted in the range from 100 to 450 psig over the steps a), b) and c).

### TECHNICAL FIELD

The present invention relates to a method of producing trimellitic acid by oxidizing pseudocumene (1,2,4-trimethylbenzene) with gas containing molecular oxygen. More particularly, the present invention is directed to a method of producing trimellitic acid by liquid-phase oxidizing pseudocumene in an acetic acid solvent under a gas atmosphere containing molecular oxygen using a combination of oxidizing catalytic ingredients selected from the group consisting of cobalt, manganese, zirconium and bromine.

### BACKGROUND ART

As well known to those skilled in the art, trimellitic acid is an intermediate of a synthetic resin, and dehydrated to produce trimellitic anhydride (TMA) used as a heat stable plasticizer, an epoxy resin hardening agent, or various additives for improving heat resistance.

A process of producing trimellitic anhydride using pseudocumene as a starting material comprises the steps of oxidizing pseudocumene to produce trimellitic acid, dehydrating trimellitic acid to produce trimellitic anhydride, refining trimellitic anhydride, and distilling a used solvent to recycle the solvent. Of these steps, the oxidizing step has the biggest effect on purity of trimellitic anhydride. The reason for this is that the impurities contained in the resulting trimellitic anhydride obtained from the dehydration step of trimellitic acid or the subsequent refining step thereto, mainly consists of organic impurities and derivatives thereof which are by-products in the oxidation of pseudocumene. Thus, in the case of producing trimellitic anhydride using trimellitic acid with low purity, it is difficult to obtain trimellitic anhydride with high purity in high yield.

Conventionally, trimellitic acid has been produced by oxidizing pseudocumene in an acetic acid solvent under a gas atmosphere containing molecular oxygen in the presence of catalysts including one or more heavy metal compounds and a bromine compound. For example, U.S. Pat. Nos. 4,537,978, 4,587,350, 4,755,622, and 4,764,639 are directed to a method of producing trimellitic acid by oxidizing pseudocumene through discontinuous two-step oxidation reaction. The first oxidation reaction is conducted at a relatively low temperature in the presence of oxidizing catalysts consisting of cobalt, manganese, zirconium and bromine. The second oxidation reaction is conducted at a relatively high temperature while adding a bromine catalyst to an oxidizing reactor, even though a temperature and compositions of pseudocumene and each catalyst may be slightly different from each patent. However, these patents are disadvantageous in that the second oxidation reaction is performed while adding the catalyst consisting of only bromine to the oxidizing reactor, thus not obtaining a sufficiently high purity of trimellitic acid.

Meanwhile, U.S. Pat. Nos. 4,845,275, 4,895,978, 4,992,579, and 5,250,724 disclose a process of producing trimellitic acid, in which undesirable production of high boiling point impurities is suppressed, and a gasification reaction of pseudocumene into carbon dioxide is prevented through complete oxidation of pseudocumene by use of catalysts comprising other metals such as lead and cerium in addition to cobalt, manganese, zirconium and bromine.

However, these patents have disadvantages in that it is not preferable to commercialize the process because the purity of trimellitic acid is not sufficiently improved and lead and cerium are used as additional catalysts, which are very harmful to human body.

### DISCLOSURE OF THE INVENTION

The present inventors have conducted extensive studies in order to avoid disadvantages of the conventional techniques, resulting in the finding that trimellitic acid is produced in high yields through three-step oxidation reaction having different temperatures, reaction times, and catalytic conditions. In other words, pseudocumene is firstly oxidized in the presence of an initial oxidizing catalytic system, secondly oxidized under the addition of an additional catalytic system to the oxidizing reactor, and then maintained for a predetermined period under the condition of increased temperature and pressure without addition of the additional catalyst to produce trimellitic acid.

Accordingly, it is an object of the present invention to provide a method for producing trimellitic acid in high purity and high yield by improving the oxidation of pseudocumene, which has the largest effect on purity and yield of trimellitic anhydride in a process of producing trimellitic anhydride.

Based on the present invention, the above object can be accomplished by a provision of a method for producing trimellitic acid through liquid-phase oxidation of pseudocumene in an acetic acid solvent under a gas atmosphere containing molecular oxygen in the presence of a catalytic system containing a combination of catalytic ingredients selected from the group consisting of cobalt, manganese, zirconium, and bromine, said oxidation of pseudocumene comprising:
a) conducting a first oxidation in the presence of an initial oxidizing catalytic system at a temperature from 120 to 200° C. for a time from 5 to 20 min in an oxidizing reactor, said initial oxidizing catalytic system comprising at least three compounds selected from the group consisting of cobalt compound, manganese compound, zirconium compound and bromine compound;
b) conducting a second oxidation in situ at a temperature from 160 to 220° C. for a time from 30 to 60 min under addition of an additional catalytic system, said additional catalytic system comprising at least two compounds selected from the group consisting of cobalt compound, manganese compound, zirconium compound, and bromine compound; and
c) completing the oxidation of pseudocumene into trimellitic acid at a temperature from 180 to 230° C. for a time from 5 to 20 min without the addition of catalysts into the reactor,
wherein a pressure is adjusted in the range from 100 to 450 psig over the steps a), b) and c)

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flow diagram schematically showing production of trimellitic acid from pseudocumene according to an embodiment of the present invention.

### BEST MODE FOR CARRYING OUT THE INVENTION

A more detailed description of a method of producing trimellitic acid will be given, below.

As described above, the present invention pertains to a method of producing trimellitic acid through liquid-phase oxidation of pseudocumene in an acetic acid solvent under a gas atmosphere containing molecular oxygen using a combination of oxidizing catalytic ingredients selected from the group consisting of cobalt, manganese, zirconium and bromine.

As such, the oxidation of pseudocumene into trimellitic acid is performed through three steps. Preferably, the present process is carried out in a discontinuous mode because the continuous mode is less favorable than the discontinuous mode in terms of the yield of trimellitic acid and the efficiency of the overall process.

In accordance with the present invention, the oxidizing catalytic system should be employed for the first and the second oxidation steps. The catalytic ingredients group suitable for such oxidizing catalytic system includes cobalt compound, manganese compound, zirconium compound, and bromine compound, and are used in the combined form thereof. The oxidizing catalytic system for the first oxidation step contains the combinations of at least three catalytic ingredients, while the oxidizing catalytic system for the second oxidation step contains the combinations of at least two catalytic ingredients.

The cobalt compound, the manganese compound, and the zirconium compound are not restricted by specific examples if being dissolved in the acetic acid. Employable are organic acid salt such as acetate, propionate, naphthenate, and octenate, hydroxide, halide (e.g. chloride and bromide), and inorganic acid salt such as borate, nitrate and carbonate, and preferably acetate, phosphate, hydroxide and bromide, and more preferably acetate.

Representatives of the bromine compound are bromine, hydrogen bromide, ammonium bromide, an alkali metal bromide such as sodium bromide, lithium bromide and potassium bromide, inorganic bromide such as cobalt bromide and manganese bromide, organic bromide such as ethane tetrabromide, acetyl bromide and benzyl bromide. Among them, sodium bromide, hydrogen bromide, cobalt bromide, manganese bromide, and ammonium bromide are preferably employed. Sodium bromide is more preferable.

An amount of cobalt to pseudocumene is 0.1 to 0.4 wt % in the initial oxidizing catalytic system, and 0 to 0.2 wt % in the additional catalytic system.

An amount of manganese to pseudocumene is 0.01 to 0.1 wt % in the initial oxidizing catalytic system, and 0.01 to 0.3 wt % in the additional catalytic system.

An amount of zirconium to pseudocumene is 0 to 0.01 wt % in the initial oxidizing catalytic system, and 0 to 0.01 wt % in the additional catalytic system.

An amount of bromine to pseudocumene is 0.01 to 0.1 wt % in the initial oxidizing catalytic system, and 0.05 to 0.5 wt % in the additional catalytic system.

If an amount of each metal compound constituting is less than the above ranges, trimellitic acid with desirable purity is not obtained due to the reduction of the oxidation rate of pseudocumene. On the other hand, where the amount is unnecessarily large, the side reaction rate is so increased as to promote the production of impurities, and losses attributable to the complete oxidation of the acetic acid or pseudocumene are problematic.

Meanwhile, as the oxidation goes on, the cobalt, manganese, and zirconium in the oxidizing catalytic system form a complex salt with the trimellitic acid, thus causing deactivation of the catalytic systems. This phenomenon makes it difficult to recycle the used catalysts. Furthermore, bromine substitutes a hydrogen atom of trimellitic acid to form bromo trimellitic acid. In addition, it is desirable to use as small an amount of catalyst as possible so as to reduce catalyst cost.

In accordance with the present invention, the first, the second and the third oxidation steps are carried out at the temperature from 120 to 200° C., from 160 to 220° C., and from 180 to 230° C., respectively. Where each reaction temperature is lower than the above temperature range, an oxidation rate of pseudocumene is so reduced that the desired reaction extent may not be obtained. On the other hand, where each temperature is higher than the above temperature range, the yield of trimellitic acid is lowered due to the complete oxidation of acetic acid or pseudocumene.

The reaction pressure should be controlled in the range of 100 to 450 psig over the three oxidation steps to maintain the acetic acid solvent in a liquid phase within the above temperature range.

According to the present invention, the partial pressure of the molecular oxygen in the reacting system needs to be maintained in such a manner that the oxygen concentration in the gas discharged from the oxidizing reactor is in the range of about 2-8 volume %. For this, the reaction time for each of the three oxidation steps is controlled in the range of 5 to 20 min, 30 to 60 min, and 5 to 20 min. If the oxygen concentration in the discharged gas is excessively high or low, the side products are increased, thus reducing the yield of trimellitic acid. In particular, it should be noted that the oxygen concentration is controlled to less than 8 volume % in order to prevent the acetic acid solvent from exploding.

A molar ratio of pseudocumene to the acetic acid solvent is 1:2 to 1:12, and preferably 1:4 to 1:10.

As described above, a portion of trimellitic acid produced at the early stage of the oxidation reaction of pseudocumene causes deactivation of the catalytic system. Accordingly, the additional catalytic system should be added to the oxidizing reactor at the second oxidation reaction step so as to obtain trimellitic acid of high purity. For similar reasons, it is preferred that the additional catalytic system is introduced to the reactor as the mixture of the catalytic ingredients, rather than as a single catalytic ingredient.

Additionally, in the case of the third oxidation reaction, it is preferred that the reaction temperature and pressure are increased and maintained without the addition of the catalysts to the reactor to suppress losses due to the complete oxidation of the solvent or pseudocumene. As a result, the oxidation of pseudocumene into trimellitic acid is completed.

Typically, the produced trimellitic acid functions to form the complex salt with the catalytic ingredients to reduce activity of the catalyst, which makes it difficult to recycle the used catalyst. However, according to the present invention, trimellitic acid of high purity may be produced using a small amount of catalyst through three-step oxidation reaction. In particular, the present inventors have noted that when the purity of trimellitic acid is 96 wt % or more, major impurities contained in the product are neither dimethlybenzenecarboxylic acid nor methylphthalic acid, but three isomers of phthalic acid. Considering the concentration of impurities in the pseudocumene acting as a reactant, it is believed that the pseudocumene is converted into one of the isomers of xylene by drop of any one of three methyl groups therein during oxidation of pseudocumene, and then oxidized to form three isomers of phthalic acid. Accordingly, the present process is capable of improving the purity of trimellitic acid by suppressing the demethylation of pseudocumene.

A better understanding of the present invention may be obtained in light of the following examples that are set forth to illustrate, but are not to be construed to limit the present invention.

### COMPARATIVE EXAMPLE 1

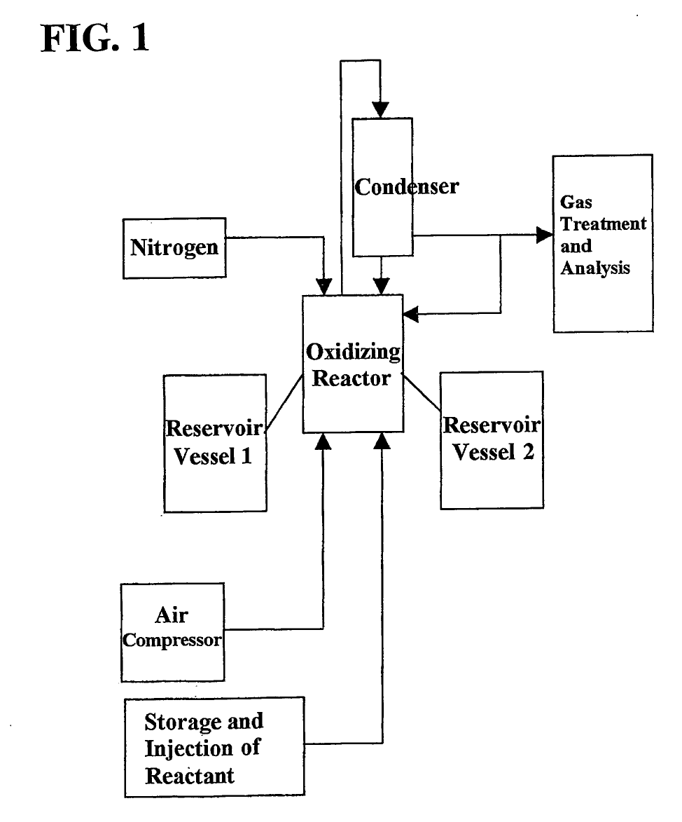

Pseudocumene was oxidized to produce trimellitic acid according to a reaction system as shown in FIG. 1. In detail, pseudocumene as a reactant and catalytic ingredients constituting a catalytic system were dissolved in acetic acid in a predetermined mixing ratio as described in Table 1, and the resulting solution was put into an oxidizing reactor. After air was removed from the oxidizing reactor using an inert nitrogen gas, a temperature in the oxidizing reactor was increased to 140° C., and the first oxidation reaction was conducted for 10 min while slowly injecting compressed air into the reactor under a constant pressure of 250 psig. The temperature and pressure in the oxidizing reactor were then increased to 205° C. and 365 psig, respectively, and the second oxidation reaction was conducted for about 50 min while adding an additional catalytic system to the reactor in a predetermined mixing ratio as described in Table 1 to accomplish oxidation of pseudocumene. At this time, a total injection amount of compressed air was controlled so as to be 1.3 times as much as a theoretical amount of air required to oxidize pseudocumene to trimellitic acid. Purity of the obtained trimellitic acid is described in Table 1.

### EXAMPLES 1-2 AND COMPARATIVE EXAMPLES 2-3

Pseudocumene as a reactant and catalytic ingredients constituting a catalytic system were dissolved in acetic acid in a predetermined mixing ratio as described in Table 1, and the resulting solution was put into the same oxidizing reactor as in Comparative Example 1. After air was removed from the oxidizing reactor using an inert nitrogen gas, a temperature in the oxidizing reactor was increased to 140° C., and the first oxidation reaction was conducted while slowly injecting compressed air into the reactor under constant pressure of 250 psig. After about 10 min, the temperature in the oxidizing reactor was increased to the temperature between 175 and 180° C., and the second oxidation reaction was then started while adding an additional catalytic system in a predetermined mixing, ratio as described in Table 1 to the reactor. Thus, the second oxidation reaction was conducted at the temperature between 190 and 195° C. for 40 min. The third oxidation reaction was conducted at 205° C. under pressure of 365 psig for 10 min while slowly reducing the amount of compressed air without the addition of any catalysts to complete oxidation of the pseudocumene. At this time, a total injection amount of compressed air was controlled to 1.3 times as much as the theoretical amount of air required to oxidize pseudocumene to trimellitic acid. Purity of the obtained trimellitic acid is described in Table 1.

TABLE 1

| | unit | Com. Ex. 1 | Exam. 1 | Exam. 2 | Com. Ex. 2 | Com. Ex. 3 |
|---|---|---|---|---|---|---|
| pseudocumene | g | 1000 | 1000 | 1400 | 1400 | 1400 |
| cobalt/pseudocumene | wt % | 0.39 | 0.39 | 0.195 | 0.195 | 0.146 |
| manganese/pseudocumene (1) | wt % | 0.064 | 0.064 | 0.032 | 0.032 | 0.024 |
| manganese/pseudocumene (2) | wt % | 0.256 | 0.256 | 0.064 | 0.064 | 0.048 |
| zirconium/pseudocumene (1) | wt % | 0.009 | 0.009 | 0.0045 | 0 | 0.0033 |
| zirconium/pseudocumene (2) | wt % | 0.018 | 0.018 | 0.0045 | 0 | 0.0033 |
| bromine/pseudocumene (1) | wt % | 0.1 | 0.1 | 0.05 | 0.05 | 0.038 |
| bromine/pseudocumene (2) | wt % | 0.8 | 0.8 | 0.2 | 0.2 | 0.15 |
| acetic acid | g | 5000 | 5000 | 3500 | 3500 | 3500 |
| trimellitic acid (purity) | % | 92.2 | 97.52 | 97.83 | 80.82 | 84.91 |

(1): first oxidation reaction
(2): second oxidation reaction

From the results of Table 1, it can be seen that purity of trimellitic acid produced through the two-step oxidation reactions according to Comparative Example 1 is very poor in comparison with the trimellitic acid produced through three-step oxidation reactions according to Example 1, even though employing the same catalytic system. Accordingly, it is confirmed that the third oxidation reaction of pseudocumene has a great influence on the overall efficiency, contrary to the teaching of the prior arts in which trimellitic acid is efficiently produced through two-step oxidation reaction. In the case of Example 2, purity of trimellitic acid is not reduced even though an amount of the catalyst to pseudocumene is half of that in Example 1.

In Comparative Example 2, the oxidation of pseudocumene is performed using the initial and additional catalytic systems not containing zirconium. As a result, the purity of the resulting trimellitic acid is greatly reduced. Accordingly, it can be seen that zirconium is required to desirably produce trimellitic acid. Additionally, in the case of Comparative Example 3, when an amount of each component constituting the catalytic systems is 75% of that of Example 2, the purity of trimellitic acid is greatly reduced due to an insufficient amount of catalytic ingredients.

Consequently, catalytic and reaction conditions of Example 2 yielding trimellitic acid with a purity of 97.82% are most preferable to oxidize pseudocumene into trimellitic acid.

### COMPARATIVE EXAMPLES 4 AND 5

Trimellitic acid was produced by oxidizing pseudocumene using a reaction system as shown in FIG. 1 in a continuous mode.

Oxidation reaction conditions of pseudocumene and the results of oxidation reaction of pseudocumene are given in Table 2. Pseudocumene was oxidized into trimellitic acid using the oxidizing catalytic systems consisting of cobalt, manganese, and bromine through two oxidation reactions having different conditions of temperature and pressure. Among plural oxidation reaction experiments, reaction conditions and purity of trimellitic acid with the highest purity are described in Table 2.

TABLE 2

| | | Com. Ex. 4 | | Com. Ex. 5 | |
|---|---|---|---|---|---|
| | | 1^{st} | 2^{nd} | 1^{st} | 2^{nd} |
| | unit | reaction | reaction | reaction | reaction |
| pseudocumene | g | 904.50 | | 2713.5 | |
| cobalt/pseudocumene | wt % | 0.86 | 0.21 | 0.86 | 0.21 |
| manganese/ | wt % | 0.44 | 0.11 | 0.44 | 0.11 |
| pseudocumene | | | | | |
| bromine/ | wt % | 1.66 | 0.40 | 1.66 | 0.40 |
| pseudocumene | | | | | |
| acetic acid | g | 17185.5 | | 51556.5 | |
| pressure | psig | 85 | 280 | 85 | 280 |
| temperature | ° C. | 141 | 201 | 142 | 208 |
| reaction time | min | 60 | 60 | 60 | 90 |
| trimellitic acid | % | | 53.43 | | 82.20 |
| (purity) | | | | | |

From the results of Table 2, it can be seen that the continuous process does not desirably accomplish the oxidation reaction of pseudocumene to trimellitic acid even though having various advantages.

### INDUSTRIAL APPLICABILITY

As described above, the present invention is advantageous in that a demethylation of pseudocumene is greatly suppressed unlike the conventional method of producing trimellitic acid through the two-step oxidation reactions, thereby improving purity of the trimellitic acid.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method for producing trimellitic acid through liquid-phase oxidation of pseudocumene in an acetic acid solvent under a gas atmosphere containing molecular oxygen in the presence of a catalytic system containing at least one selected from the group consisting of cobalt, manganese, zirconium and bromine, said oxidation of pseudocumene comprising:
a) conducting a first oxidation in the presence of an initial oxidizing catalytic system at a temperature from 120 to 200° C. for a time from 5 to 20 min in an oxidizing reactor, said initial oxidizing catalytic system comprising at least three compounds selected from the group consisting of cobalt compound, manganese compound, zirconium compound and bromine compound;
b) conducting a second oxidation in situ at a temperature from 160 to 220° C. for a time from 30 to 60 min under addition of an additional catalytic system, said additional catalytic system comprising at least two compounds selected from the group consisting of cobalt compound, manganese compound, zirconium compound, and bromine compound; and
c) completing the oxidation of pseudocumene into trimellitic acid at a temperature from 180 to 230° C. for a time from 5 to 20 min without the addition of catalysts into the reactor,
wherein a pressure is adjusted in the range from 100 to 450 psig over the steps a), b) and c).

2. The method as set forth in claim 1, wherein an amount of cobalt is 0.1 to 0.4 wt %, an amount of manganese is 0.01 to 0.1 wt %, an amount of zirconium is 0 to 0.01 wt %, and an amount of bromine is 0.01 to 0.1 wt % based on a weight of pseudocumene in the initial oxidizing catalytic system.

3. The method as set forth in claim 1, wherein an amount of cobalt is 0 to 0.2 wt %, an amount of manganese is 0.01 to 0.3 wt %, an amount of zirconium is 0 to 0.01 wt %, and an amount of bromine is 0.05 to 0.5 wt % based on a weight of pseudocumene in the additional catalytic system.

4. The method as set forth in claims 1, wherein the oxygen concentration in the gas discharged from the oxidizing reactor is maintained in the range of 2 to 8 volume %.

5. The method as set forth in claim 1, wherein a molar ratio of the pseudocumene to the acetic acid solvent is 1:2 to 1:12.

6. The method as set forth in claim 1, wherein the step c) is performed under the condition of higher temperature and pressure than the step b).

7. The method as set in claim 1, wherein the method is carried out in a discontinuous mode.
